(19) United States
(12) Patent Application Publication
(10) Pub. No.: US 2009/0264926 A1
(43) Pub. Date: Oct. 22, 2009

(54) Spinal Fixation System

(75) Inventors: Harold Sparr Taylor, Memphis, TN (US); Douglas N. Baker, Collierville, TN (US); Marco Capote, Louisville, CO (US); Dennis Crandall, Mesa, AZ (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/106,286

(22) Filed: Apr. 19, 2008

(51) Int. Cl. A61B 17/70; A61B 17/04

(52) U.S. Cl. 606/246; 606/250; 606/301; 606/264

(57) ABSTRACT

Embodiments of a spinal implant system include a fixation element, a connector body, and a compression member. The fixation element has a bone engaging portion, a pivoting portion, and a hinge portion. The hinge portion permits pivotal motion of the pivoting portion relative to the bone engaging portion. Additionally, the connector body defines a channel configured for receipt of an elongate member and defines an elongate slot configured for receipt of the pivoting portion of the fixation element. The pivoting portion is positionable in any one of a plurality of positions along the elongate slot. The compression member is adapted to engage the pivoting portion of the fixation element and press against the connector body to lock the pivoting portion at a desired angular position relative to the bone engaging portion and lock the connector body to the fixation element at the desired position along the elongate slot.

The present disclosure broadly concerns spinal fixation systems and generally relates to a multi-plane adjustment system involving a pivoting screw assembly. The system can be useful for correction of spinal injuries or deformities.

The present disclosure generally relates to a spinal fixation system useful for correction of spinal injuries or deformities. More specifically, but not exclusively, the present disclosure concerns spinal fixation systems allowing for pivotal motion of bone screws and lateral adjustment of spinal rod connectors relative to the bone screws, and providing locking mechanisms for locking the bone screws and connectors at desired positions.

In the realm of orthopedic surgery, it is well known to use implants to fix the position of bones. In this way, the healing of a broken bone can be promoted, and malformations or other injuries can be corrected. For example, in the field of spinal surgery, it is well known to place such implants into vertebrae for a number of reasons, including (a) correcting an abnormal curvature of the spine, including a scoliotic curvature, (b) to maintain appropriate spacing and provide support to broken or otherwise injured vertebrae, and (c) perform other therapies on the spinal column.

Implant and connection systems may include several pieces, which may be associated with only specific other pieces. Bone screws, hooks, clamps or other fixation devices can be connected or adjoined to a particular bone as a connection between the bone and the connection system, which can include a support and/or stabilizing member such as a spinal rod. In such a system, a series of two or more screws may be inserted into two or more vertebrae to be instrumented. A rod is then placed within or coupled to the screws, or is placed within a connecting device that links the rod and a screw, and the connections are tightened. In this way, a rigid supporting structure is fixed to the vertebrae, with the rod providing the support that promotes correction or healing of the vertebral malformation or injury by keeping the vertebrae in a particular position.

A spinal implant system or other similar system may have anchors that can be positioned at a number of angles with respect to the vertebrae or spinal rods. Such a feature allows easier placement of implant systems or correction of positioning of an implant system, in that the bone anchors need not be precisely positioned in angular relation with respect to the vertebrae or spinal rods. Rather, with a multi-axial capability, holes can be drilled in a bone at a convenient location and/or angle, for example, and screws can be inserted therein.

A need remains for spinal fixation systems allowing for multi-plane adjustment and which link elongated member(s) to fixation device(s) and allow for pivoting and other adjustment capabilities of the components of the system.

### BRIEF DESCRIPTION OF THE DRAWINGS

FIG. 1 is a side view of an implant system according to one embodiment.

FIG. 2A is a front view of components of the implant system according to the embodiment shown in FIG. 1.

FIG. 2B is a side view of components of the implant system according to the embodiment shown in FIG. 1.

FIG. 3 is a perspective view of a component of the implant system according to the embodiment shown in FIG. 1.

FIG. 4 is a side view of an implant system according to another embodiment.

FIG. 5 is an exploded view of the implant system according to the embodiment shown in FIG. 4.

FIG. 6 is a perspective view of components of the implant system according to the embodiment shown in FIGS. 4 and 5.

FIG. 7 is a side view of an implant system according to yet another embodiment.

FIG. 8 is a partial exploded view of the implant system according to the embodiment shown in FIG. 7.

FIG. 9 is front view of the implant system according to the embodiment shown in FIGS. 7 and 8.

### DESCRIPTION OF THE ILLUSTRATED EMBODIMENTS

For the purpose of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated device, and such further applications of the principles of the disclosure as illustrated therein, being contemplated as would normally occur to one skilled in the art to which the disclosure relates.

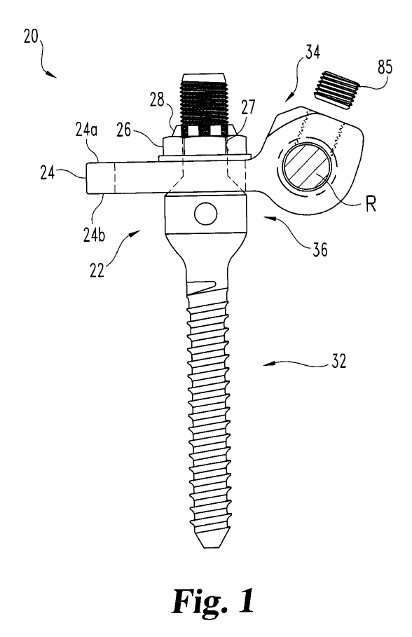

Referring generally to FIGS. 1-3, there is shown an embodiment of a spinal fixation system 20. System 20 generally includes a fixation device or anchor (e.g. a pivoting bone screw or bolt 22), a connector 24 and a nut 26 in that embodiment. Connector 24, having a top surface 24a and a bottom surface 24b, can connect an elongate member, such as a spinal rod, a bar, or other such orthopedic construct, to anchor 22. The elongate member may have any of a number of desired lengths. Bone screw 22 includes a bone engaging portion 32, a pivoting post portion 34 and a hinge portion 36 between the bone engaging portion 32 and pivoting post portion 34. Hinge portion 36 permits pivotal motion of post portion 34 relative to bone engaging portion 32.

In certain embodiments, bone engaging portion 32 can be threaded to engage a bone structure, such as a vertebral body, and solidly anchor screw 22 to the bone structure. In such embodiments, bone engaging portion 32 can include coarse threads readily adapted for solid fixation within the cancellous bone of a vertebral body and can terminate in a tapered tip to assist in the gradual engagement and advancement of the threads into the vertebral body. In alternative embodiments, it should be appreciated that bone engaging portion 32 can have a variety of configurations and/or can be a hook or other such appropriate bone engaging structure. Post portion 34 can also be threaded to engage nut 26 or other such threaded items. In certain embodiments, post portion 34 includes machine threads upon which a surgeon may attach an item, such as a clamp. Nut 26 defines a threaded inner hole 27 to threadedly couple with post portion 34. Nut 26 is operable to lock pivoting post portion 34 at a desired angular position relative to bone engaging portion 32. Turning and tightening nut 26 thereby locks connector 24 at the same desired angular position as post portion 34. In certain embodiments, nut 26 can include locking tabs 28 to further secure and lock system 20 at desired positions.

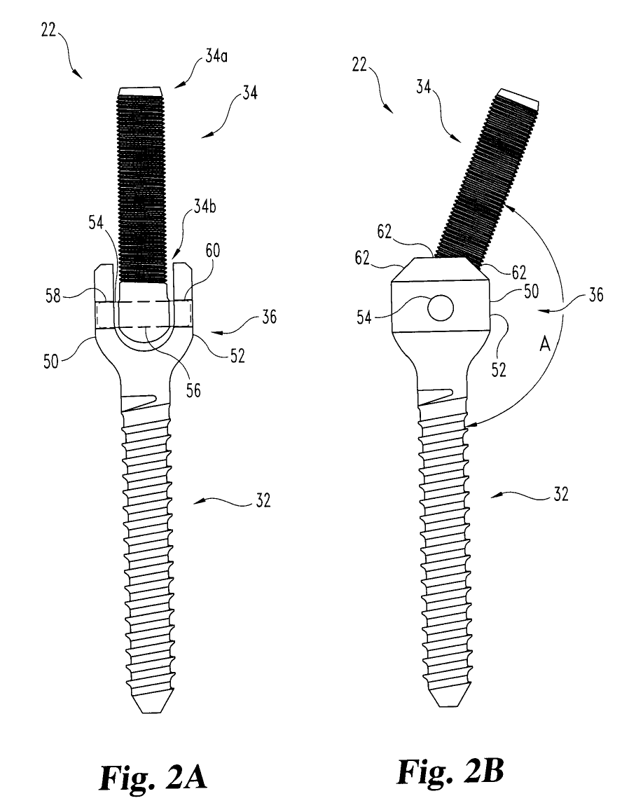

FIG. 2A is a front view of anchor 22 according to the embodiment shown in FIG. 1. Post portion 34 includes a proximal end 34a and a distal end 34b, with distal end 34b being operably coupled with hinge portion 36. Hinge portion 36 of screw 22 can include extensions 50 and 52. In certain embodiments, extensions 50 and 52 are generally parallel and extend upward from bone engaging portion 32. Extensions 50 and 52 are spaced apart to generally define a channel to receive distal end 34b of post portion 34. The width of the space between extensions 50 and 52 may be larger than the diameter of post portion 34. Hinge portion 36 of screw 22 can also include a hinge pin 54. In certain embodiments, post portion 34 rotates about hinge pin 54 relative to bone engaging portion 32. Additionally, post portion 34 includes an aperture 56 and extensions 50 and 52 include apertures 58 and 60, respectively, to receive hinge pin 54. In certain embodiments, hinge pin 54 can be received and maintained in apertures 56, 58, and 60 in an interference fit relationship. During assembly, pin 54 can be inserted through one aperture 58 or 60, through aperture 56 in post portion 34, and through the other of aperture 58 or 60.

FIG. 2B is a side view of anchor 22 of the embodiment shown in FIG. 1. As illustrated, post portion 34 can pivot about hinge 54 relative to bone engaging portion 32. In the illustrated embodiment, post portion 34 is pivoted to a position such that an angle A exists between post portion 34 and bone engaging portion 32. Extensions 50 and 52 of hinge portion 36 can include three generally straight segments 62 to contact connector 24. In other embodiments, straight segments 62 can number more or less than three. Additionally, in other embodiments, extensions 50 and 52 can include generally curved convex or conical upper surfaces to contact connector 24. In the illustrated embodiment, straight segments 62 are configured to contact bottom surface 24b of connector 24. Straight segments 62 can correspond to angular position(s) of post portion 34 relative to bone engaging portion 32.

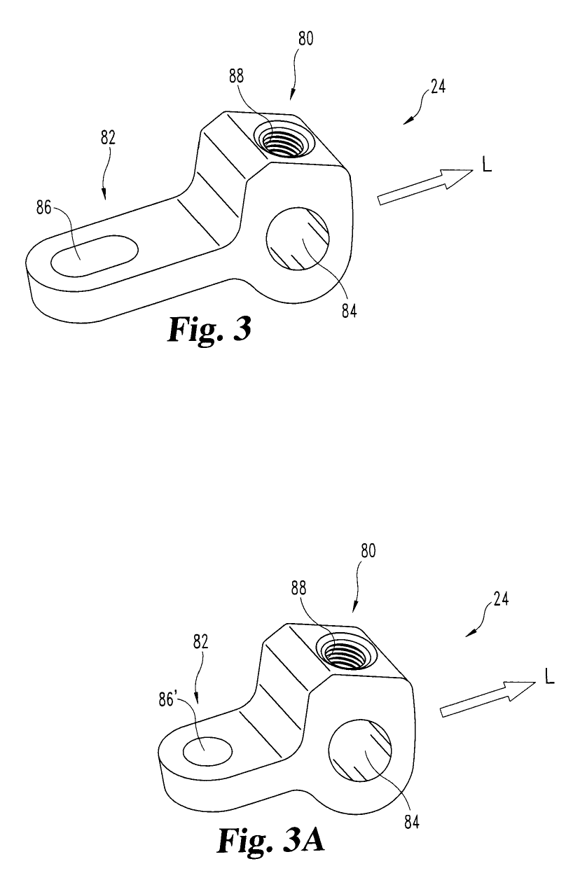

FIG. 3 is a perspective view of connector 24 according to the embodiment shown in FIG. 1. In the illustrated embodiment, connector 24 extends along a longitudinal axis L and includes a elongated member engaging portion 80 and an anchor engaging portion 82. Portion 80 defines a channel 84 configured for receipt of an elongate member, such as a spinal rod. In certain embodiments, channel 84 is generally perpendicular to longitudinal axis L. Portion 82 defines an elongate slot 86 in this embodiment, which is configured in this embodiment for receipt of post portion 34 of bone screw 22. In certain embodiments, slot 86 extends generally along longitudinal axis L.

Additionally, portion 80 can define a through hole 88 in communication with channel 84 configured to receive a retaining member, such as a set screw 85. In certain embodiments, hole 88 can include internal threaded portion(s) to be threadedly coupled with the retaining member to secure an elongate member in channel 84. In such embodiments, the internal threaded portions can include reverse angle threads, i.e. a thread in which the forward face points as disclosed in U.S. Pat. No. 6,296,642, incorporated herein by reference in its entirety. In certain embodiments, retaining member 85 is a generally cylindrical set screw with external threads, but may alternatively be another type of holding or locking mechanism. However, it should be appreciated that the elongate member can be secured in channel 84 by other appropriate methods. In certain embodiments, channel 84 may be non-circular in shape to better allow for a retaining member, such as a set screw, to tightly retain an elongate member in channel 84. It should be appreciated that channel 84 can be configured and shaped differently as would generally occur to one skilled in the art.

In certain embodiments, slot 86 includes a sufficient length along longitudinal axis L to allow for adjustment of post portion 34 and positioning of post portion 34 at any one of a plurality of positions along slot 86. The available positioning of post portion 34 at a plurality of positions along slot 86 thereby allows for the relative positioning of an elongate member received in channel 84 at a plurality of positions relative to bone screw 22. Nut 26 is operable to lock pivoting post portion 34 at a desired position along slot 86.

Referring generally to FIGS. 1-3, the operation and use of system 20 will be described with reference to a surgical procedure involving a section of spine. It will be appreciated that other uses of system 20 in other surgical procedures can be made.

To treat the condition or injury of the patient, the surgeon obtains access to the surgical site in a manner well known in the art, e.g. through incision and retraction of tissues. Once access to the surgical site has been obtained, e.g. via an opening such as a midline incision above the affected area, with tissue being resected laterally to the transverse process, or by other surgical procedure, the surgeon may connect one or more implants, such as bone screws, to adjacent or nearby vertebrae that require compression or distraction in order to relieve or improve their condition. For example, pilot holes in vertebrae, e.g. in pedicles, may be made, and anchors (e.g. screws 22) may be inserted into or otherwise connected to two or more vertebrae. In one embodiment, once an appropriate access to a surgical site is obtained, system 20 can be inserted to the surgical site, and may be placed in a desired position at or adjacent certain vertebrae. For example, screw 22 may be inserted into a bony structure, such as a vertebra, at a desired position. Threaded portion 32 of each such screw 22 can be threaded into bone to a desired depth, and connector 24 may be placed on screw 22 so that portion 34 extends through slot 86.

An elongate member (e.g. rod R) is placed in channel 84 of connector 24 either before or after connection of connector 24 to screw 22 or placement of system 20 near vertebrae. Retaining member 85 can be received in hole 88 of connector 24 to loosely hold elongate member R therein, while allowing translational and rotational adjustability. Elongate member R can later be received in another connecting assembly with a bone screw or other implant inserted into another vertebra to secure a section of vertebrae.

In certain embodiments, a user of system 20 can pivot post portion 34 to a desired angular position relative to bone engaging portion 32. In the illustrated embodiment, bottom surface 24b of connector 24 can contact one or more of straight segments 62 of extensions 50 and 52 corresponding to a desired angular position of post portion 34. In alternative embodiments, connector 24 engages extensions 50 and 52 in other manners as would occur to one skilled in the art. Additionally, a user can position post portion 34 at a desired location along slot 86 of connector 24. Prior to locking, connector 24 can pivot with respect to portion 32 of anchor 22, or can be moved around an axis of portion 34 of anchor 22. Connector 24 can also be translated along slot 86 with respect to screw 22. Connector 24 can also be pivoted with respect to or translated along elongated member R.

After multi-plane adjustment of system 20, it can be locked to fix the relative positions of elongated member R, screw 22 and connector 24. In certain embodiments, nut 26 can engage post portion 34 by threading nut 26 down post portion 34 to thereby lock system 20 at the desired position by clamping connector 24 to hinge portion 36 of bone screw 22. In certain embodiments, locking tabs 28 can be used to further engage post portion 24 and lock system 20 and the desired positions. Either before or after locking of system 20, a user can insert portion 32 of anchor 22 in a vertebral body and can insert elongate member R in channel 84. Set screw or other retaining member 85 is tightened in hole 88 to securely hold member R in connector 24. System 20 may generally be assembled prior to use in a surgical procedure. However, it should be appreciated that system 20 can be assembled during the surgical procedure.

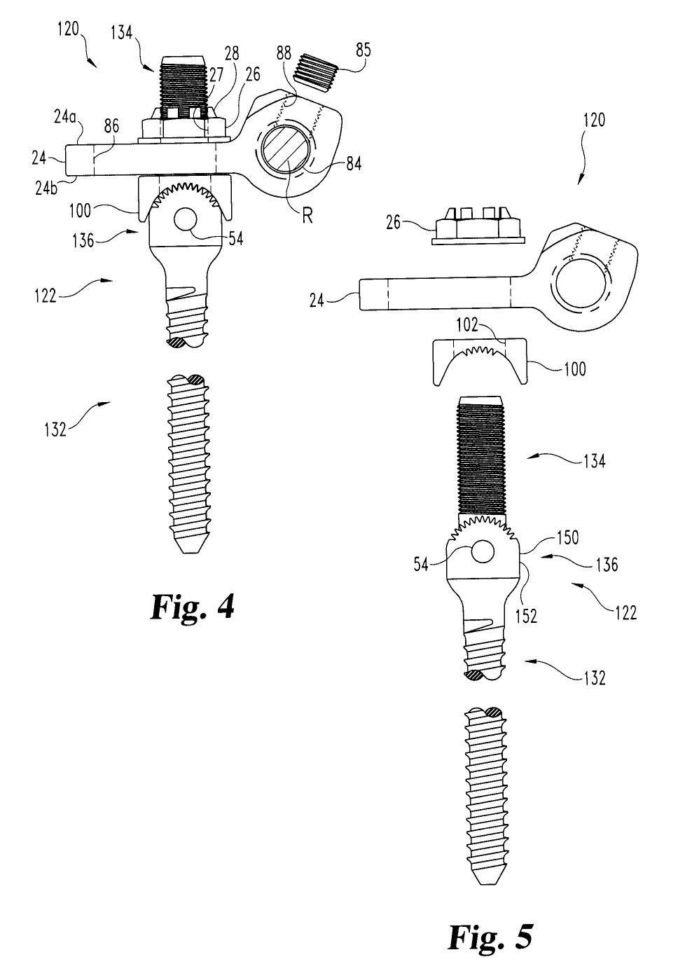

FIG. 4 illustrates a system 120 according to another embodiment where like reference numerals refer to like features previously discussed. System 120 generally includes a pivoting anchor such as bone screw 122, connector 24 and nut 26. Connector 24 connects an elongate member, such as a spinal rod R, to screw 122. System 120 can further include a locking piece 100 positionable below connector 24 adjacent bottom surface 24b.

FIG. 5 is an exploded view of system 120. As illustrated, locking piece 100 defines a hole 102 configured to receive the post portion of screw 122. Screw 122 can include bone engaging portion 132, pivoting post portion 134 and hinge portion 136 between portion 132 and pivoting post portion 134. Similar to anchor 22, hinge portion 136 of screw 122 permits pivotal motion of post portion 134 relative to engaging portion 132. In certain embodiments, bone engaging portion 132 can be threaded to engage a vertebra. Additionally in certain embodiments, post portion 134 can be threaded to engage nut 126 or other such items.

As illustrated, hinge portion 136 of bone screw 122 includes extensions 150 and 152. In certain embodiments, extensions 150 and 152 are generally parallel and extend upward from bone engaging portion 132. Additionally, hinge portion 136 can include a hinge pin 54 about which pivoting post portion 134 rotates relative to bone engaging portion 132. Similar to screw 22, post portion 134 and extensions 150 and 152 can include holes to receive hinge pin 54.

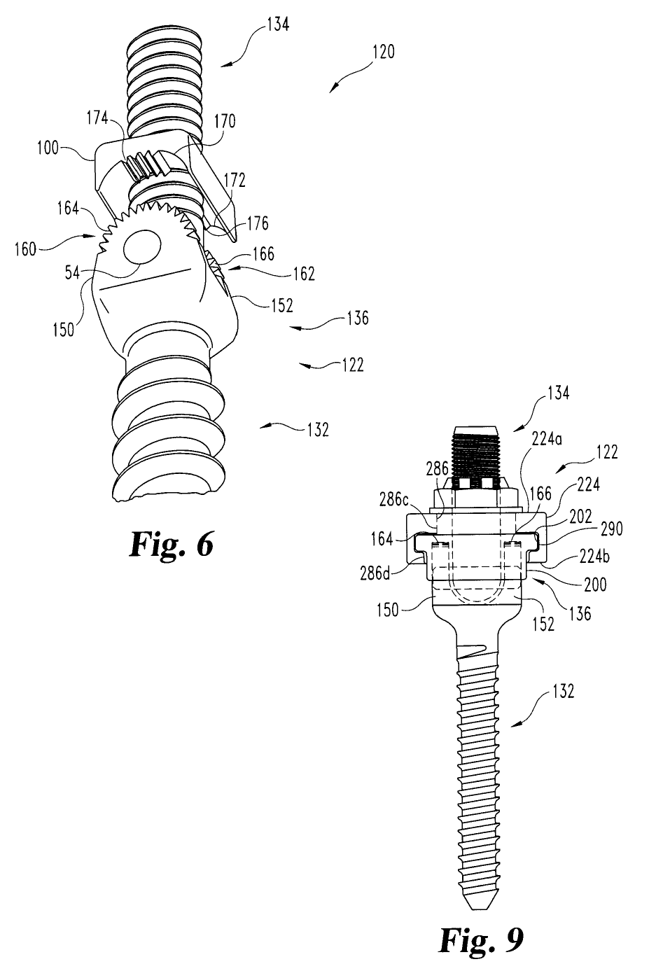

FIG. 6 illustrates select components of system 120, including extensions 150 and 152 and locking piece 100. Extensions 150 and 152 generally include upper convex surfaces 160 and 162, respectively, having splines 164 and 166, respectively. A bottom surface of locking piece 100 includes two generally parallel concave surfaces 170 and 172 having splines 174 and 176, respectively, to engage splines 164 and 166 on extensions 150 and 152 at a plurality of positions. The engagement of the sets of splines at various positions corresponds to various angular positions of post portion 134 relative to bone engaging portion 132. In alternative embodiments, the splines on the extensions and the locking piece are absent, with generally curved concave and convex surfaces of the extensions and the locking piece being in engagement.

Referring generally to FIGS. 4-6, the operation and use of system 120 will be described with reference to a surgical procedure involving a section of spine. It will be appreciated that other uses of system 120 in other surgical procedures can be made. The surgeon obtains access to the surgical site as previously described in greater detail. In one embodiment, once an appropriate access to a surgical site is obtained, system 120 can be inserted to the surgical site, and may be placed in a desired position at or adjacent certain vertebrae. Thereafter, screw 122 is inserted into a bony structure, such as a vertebra, at a desired position. Threaded engaging portion 132 can be threaded into bone to a desired depth.

An elongate member (e.g. rod R) is placed in channel 84 of connector 24 either before or after connection of connector 24 to anchor 122 or placement of system 120 near the vertebrae. A retaining member (e.g. set screw 85) can be received in hole 88 of connector 24 to loosely hold elongate member R therein, while allowing translational and rotational adjustability. Elongate member R can later be received in another connecting assembly with a bone screw or other implant inserted into another vertebra to secure a section of vertebrae.

In certain embodiments, a user of system 120 can pivot post portion 134 to a desired angular position relative to bone engaging portion 132. In the illustrated embodiment, bottom surface 24b of connector 24 contacts an upper surface of locking piece 100. Additionally, splines on locking piece 100 contact splines on extensions 150 and 152 at a desired position corresponding to a desired angular position of post portion 134. In alternative embodiments, locking piece 100 engages extensions 150 and 152 in other manners. Additionally, similar to system 20, a user can position post portion 134 at a desired location along slot 86 of connector 24. Connector 24 can pivot with respect to portion 132 and along with portion 134, and can translate along slot 86 with respect to anchor 122. Connector 24 can also be pivoted and/or translated with respect to elongated member R.

After multi-plane adjustment of system 120, it can be locked so as to fix elongated member R, connector 24 and screw 122 with respect to each other. In certain embodiments, engagement of nut 26 to post portion 134 by threading nut 26 down post portion 134 operates to lock system 120 at the desired position by clamping connector 24 between nut 26 and locking piece 100, thereby clamping locking piece 100 to hinge portion 136 of bone screw 122. Additionally, similar to system 120, either before or after locking of system 120, a user can insert bone engaging portion 132 in a vertebra and can insert an elongate member R in channel 84 of connector 24. A retaining member (e.g. set screw 85) may be tightened in hole 88 to securely hold elongated member R to connector 24. System 120 may generally be assembled prior to use in a surgical procedure. However, it should be appreciated that system 120 can be assembled during the surgical procedure.

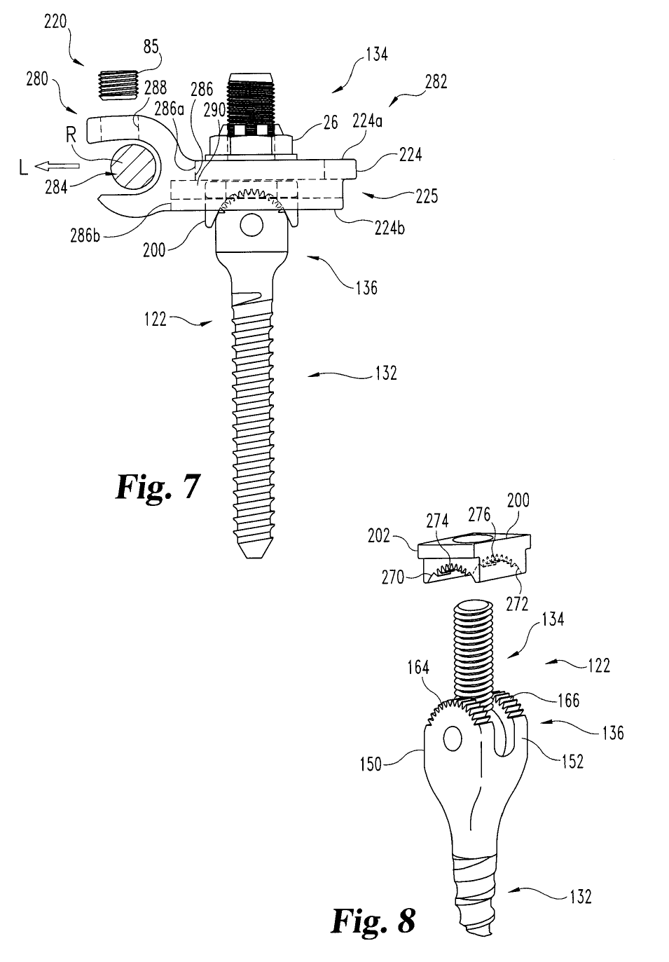

FIG. 7 illustrates a system 220 according to another embodiment where like reference numerals refer to like features previously discussed. System 220 generally includes a pivoting anchor such as bone screw 122, a connector 224 and nut 26. Connector 224 is operable to connect an elongate member, such as a spinal rod R, to bone screw 122 and includes a top surface 224a and a bottom surface 224b. System 220 further includes a locking piece 200 positionable within or along connector 224. In the illustrated embodiment, connector 224 includes a recessed area 225 to permit easier insertion of locking piece 200 within connector 224.

As illustrated connector 224 can extend along a longitudinal axis L and can include an elongated member engaging portion 280 and an anchor engaging portion 282. Portion 280 defines a substantially C-shaped channel 284 configured for receipt of an elongate member, such as a spinal rod R. In certain embodiments, channel 284 is generally perpendicular to longitudinal axis L. In the illustrated embodiment, channel 284 is a side loading spinal rod channel. However, it should be appreciated that channel 284 can be sized and/or configured differently as would occur to one skilled in the art. Additionally, rod engaging portion 280 can define a through hole 288 in communication with channel 284 configured to receive a retaining member 285, such as a set screw. The retaining member is operable to secure an elongate member in channel 284.

Bone screw engaging portion 282 defines an elongate slot 286 configured for receipt of post portion 134 of bone screw 122. In certain embodiments, slot 286 extends generally along or parallel to longitudinal axis L. Bone screw engaging portion 282 can also define longitudinal grooves 290 adjacent slot 286 configured to engage locking piece 200 (see FIG. 9). In the illustrated embodiment, grooves 290 extend generally along or parallel to longitudinal axis L, with one groove 290 on each side of slot 286. In certain embodiments, longitudinal grooves 290 run substantially the entire length of each longitudinal side of slot 286.

Similar to slot 86, slot 286 includes a sufficient length along longitudinal axis L to allow for adjustment of post portion 134 and positioning of post portion 134 at any one of a plurality of positions along slot 286. In certain embodiments, slot 286 includes an upper section 286a having a first length and a lower section 286b having a second length, with upper section 286a adjacent top surface 224a of connector 224 and lower section 286b adjacent bottom surface 224b of connector 224. Additionally, in certain embodiments, lower section 286b includes a greater length along longitudinal axis L than upper section 286a to increase the plurality of positions post portion 134 can occupy in slot 286. The variable positioning of post portion 134 in slot 286 allows for the positioning of an elongate member received in channel 284 at a plurality of distances or positions relative to bone screw 122. In certain embodiments, nut 26 is also operable to lock pivoting post portion 134 at the desired position within slot 286.

FIG. 8 is a partial exploded view of system 220. In certain embodiments, locking piece 200 is integral with connector 224 (see FIG. 7). Locking piece 200 can be positioned in slot 286 and can include extensions, such as fingers 202, to slidably engage channels 290. Similar to locking piece 100, a bottom surface of locking piece 200 includes two generally parallel concave surfaces 270 and 272 having splines 274 and 276, respectively, to engage splines 164 and 166 on extensions 150 and 152 at a plurality of positions. In alternative embodiments, the splines are absent and the concave and convex surfaces of locking piece 200 and extensions 150 and 152 are slidably engaged. The engagement of sets of splines at various positions corresponds to the various angular positions of post portion 134 relative to bone engaging portion 132.

FIG. 9 illustrates a front view of system 220. As illustrated, locking piece 200 can include one or more fingers 202 on opposite sides of locking piece 200 to be slidably received in grooves 290. In the illustrated embodiment, fingers 202 are generally rectangular in cross-sectional shape. However, it should be appreciated that fingers 202 can be otherwise sized and configured. Slot 286 includes an upper width section 286c adjacent top surface 224a of connector 224 and a lower width section 286d adjacent bottom surface 224b of connector 224. In certain embodiments, lower section 226d includes a width greater than upper section 226c to accommodate loading and translation of locking piece 200 in slot 286. Locking piece 200 can be positioned at a plurality of positions along slot 286 thereby positioning post portion 134 at one of a plurality of positions along slot 286.

Referring generally to FIGS. 7-9, the operation and use of system 220 will be described with reference to a surgical procedure involving a section of spine. It will be appreciated that other uses of system 220 in other surgical procedures can be made. The surgeon obtains access to the surgical site as previously descried in greater detail. In one embodiment, once an appropriate access to a surgical site is obtained, system 220 can be inserted to the surgical site, and may be placed in a desired position at or adjacent certain vertebrae. For example, screw 122 may be inserted into a bony structure, such as a vertebra, at a desired position. Threaded portion 132 of each such anchor 122 can be threaded into bone to a desired depth.

An elongate member (e.g. rod R) is placed in channel 84 of connector 224 either before or after connection of connector 224 to screw 122 or placement of system 220 near the vertebrae. A retaining member (e.g. set screw 85) can be received in hole 288 of connector 224 to loosely hold elongate member R therein, while allowing translational and rotational adjustability. Elongate member R can later be received in another connecting assembly with a bone screw or other implant inserted into another vertebra to secure a section of vertebrae.

In certain embodiments, a user of system 220 can pivot post portion 134 to a desired angular position relative to bone engaging portion 132. In certain embodiments, splines on locking piece 200 can contact splines on extensions 150 and 152 at a desired position corresponding to a desired angular position of post portion 134. In alternative embodiments, locking piece 200 engages extensions 150 and 152 in other manners as would occur to one skilled in the art. Connector 224 can pivot with respect to portion 132 and along with portion 134, and can translate along slot 286 with respect to anchor 122. Connector 224 can also be pivoted and/or translated with respect to elongated member R.

Similar to systems 20 and 120, a user of system 220 can position post portion 134 at a desired location along slot 286 of connector 224 via the sliding engagement of locking piece 200 within connector 224. The positioning of post portion 134 along slot 286 corresponds to the positioning of locking piece 200 along slot 286 by the sliding of fingers 202 along grooves 290.

After multi-plane adjustment of system 220, it can be locked so that elongate member R, connector 224 and anchor 122 are fixed with respect to each other. In certain embodiments, the engagement of nut 26 to post portion 134 by threading nut 26 down post portion 134 thereby locks system 20 at the desired positions by clamping connector 224 and locking piece 200 to hinge portion 136 of bone screw 122. Additionally, similar to systems 20 and 120, a user of system 220 can insert bone engaging portion 132 in a vertebral body and can insert an elongate member in side loading channel 284. System 220 may generally be assembled prior to use in a surgical procedure. However, it should be appreciated that system 220 can be assembled during the surgical procedure.

The parts, features and steps discussed above may be interchanged with each other or among embodiments. Accordingly, anchor 22 may be used with system 220, as one example. The various components of systems 20, 120, and 220 are composed of biocompatible materials such as titanium, stainless steel, certain ceramics or plastics, or others.

While the disclosure has been illustrated and described in detail in the drawings and foregoing description, the same is to be considered as illustrative and not restrictive in character. It should be understood that only the preferred embodiments have been shown and described and that all changes and modifications that come within the spirit of the disclosure are desired to be protected.

What is claimed is:

1. A spinal implant system, comprising:
a fixation element having a threaded bone engaging portion, a pivoting portion, and a hinge portion between said bone engaging portion and said pivoting portion to permit pivotal motion of said pivoting portion relative to said bone engaging portion, whereby said pivoting portion is positionable in any one of a plurality of angular positions relative to said bone engaging portion;
a connector body having an elongate member engaging portion and a fixation element engaging portion, wherein said elongate member engaging portion defines a channel configured for receipt of an elongate member, and said fixation element engaging portion defines an elongate slot configured for receipt of said pivoting portion of said fixation element, wherein said pivoting portion is positionable in any one of a plurality of positions along said elongate slot; and
a compression member adapted to engage said pivoting portion of said fixation element and press against said connector body, to thereby lock said pivoting portion at a desired angular position relative to said bone engaging portion and lock said connector body to said fixation element.

2. The spinal implant system of claim 1, wherein said connector body has a longitudinal axis and said elongate slot extends parallel to said longitudinal axis, wherein said elongate slot includes a length along said longitudinal axis to allow for lateral adjustment of said connector body relative to said fixation element, wherein said connector body is positionable adjacent said hinge portion of said fixation element when said pivoting portion is received in said elongate slot.

3. The spinal implant system of claim 1, wherein said pivoting portion is threaded, said compression member is a nut, and said nut includes an inner threaded surface defining a hole, and said nut is adapted to be threaded onto said pivoting portion and down onto said fixation element engaging portion of said connector body, whereby said threaded pivoting portion is received in said hole.

4. The spinal implant system of claim 1, wherein said hinge portion includes a hinge member and two lateral extensions extending from said bone engaging portion, each of said extensions and said pivoting portion of said fixation element defining a hole for passage of said hinge member.

5. The spinal implant system of claim 4, wherein each of said extensions include a top surface having two or more straight segments to contact a lower surface of said fixation element engaging portion of said connector body, corresponding to pivotal motion of said pivoting portion.

6. The spinal implant system of claim 1, comprising a hole defined by said elongate member engaging portion in communication with said channel configured to receive a retaining member to secure an elongate member in said channel.

7. A spinal implant system, comprising:
a bone screw having a threaded bone engaging portion, a threaded post portion, and a hinge portion between said bone engaging portion and said post portion, wherein said post portion is configured to pivot relative to said bone engaging portion, wherein said hinged portion includes two generally parallel side extensions extending from said bone engaging portion, each of said extensions having a generally convex upper surface;
a locking piece defining a hole therethrough configured to receive said post portion and having a top and bottom surface, wherein said bottom surface includes two generally parallel concave surfaces configured to engage said convex upper surfaces of said extensions at two or more positions;
a connector body configured to engage and connect said bone screw and a spinal rod; and
a nut adapted to threadingly engage said post portion of said fixation element and be threaded down onto said connector body, to thereby lock said post portion at a desired angular position relative to said bone engaging portion and lock said connector body to said bone screw.

8. The spinal implant of claim 7, wherein said connector body has a longitudinal axis, and a rod engaging portion and an adjustment portion, wherein said rod engaging portion defines a channel extending generally perpendicular to said longitudinal axis and configured for receipt of said spinal rod, and said adjustment portion defines a slot extending along said longitudinal axis and configured for receipt of said post portion of said fixation element.

9. The spinal implant system of claim 8, wherein said slot extends a sufficient length along said longitudinal axis to allow for lateral adjustment of said connector body relative to said bone screw, thereby allowing positioning of said post portion of said bone screw at a plurality of positions along said slot.

10. The spinal implant system of claim 8, comprising a hole defined by said rod engaging portion in communication with said channel configured for receipt of a set screw to secure a spinal rod in said channel.

11. The spinal implant system of claim 8, wherein said locking piece cooperates with said connector body, said locking piece having one or more extensions on two parallel sides and said connector body defining two parallel channels adjacent two parallel sides of said slot, wherein said extensions are configured to be slidably received in said channels.

12. The spinal implant of claim 7, comprising a set of splines disposed on each of said concave surfaces of said bottom surface of said locking piece and each of said convex upper surfaces of said extensions, wherein said splines on said concave surfaces of said locking piece are configured to cooperate with and engage said splines on said convex upper surfaces of said extensions at said two or more positions corresponding to pivotal motion of said post portion.

13. The spinal implant system of claim 7, wherein said nut includes an inner threaded surface defining a hole, said inner threaded surface being configured to engage said threaded post portion whereby said threaded post portion is received in said hole.

14. The spinal implant system of claim 7, wherein said hinge portion includes a hinge post, each of said extensions and said post portion defining a hole for passage of said hinge post.

15. A spinal implant system, comprising:
a bone screw including a bone engaging portion having a distal end and a proximal end, a pivoting post portion having a distal end and a proximal end, and a hinge pin configured to engage said proximal end of said bone engaging portion and said distal end of said pivoting post portion to pivotally couple said bone engaging portion and said pivoting post portion, wherein said post portion is configured to pivot about said hinge pin relative to said bone engaging portion, wherein said bone engaging portion includes two generally parallel side extensions adjacent said proximal end of said bone engaging portion, each of said extensions having a generally convex upper surface, each of said extensions and said post portion including a hole configured for receipt of said hinge pin;
a locking piece defining a hole therethrough configured to receive said post portion and having a top and bottom surface, wherein said bottom surface includes two generally parallel concave surfaces configured to engage said convex upper surfaces of said extensions at two or more positions;
a connector body having a longitudinal axis, and a rod engaging portion and a bone screw engaging portion, wherein said rod engaging portion defines a channel extending generally perpendicular to said longitudinal axis and configured for receipt of a spinal rod, and said bone screw engaging portion defines a slot extending along said longitudinal axis and configured for receipt of said post portion of said bone screw, wherein said connector body defines two longitudinal grooves adjacent said slot and extending substantially the length of said slot along said longitudinal axis, wherein said locking piece includes one or more fingers on two parallel sides of said locking piece configured to be slidably received in said grooves; and
a nut adapted to be threaded onto said post portion of said bone screw and down onto said bone screw engaging portion of said connector body, to thereby lock said post portion at a desired position relative to said bone engaging portion and lock said connector body to said bone screw.

16. The spinal implant system of claim 15, comprising a set of splines disposed on each of said concave surfaces of said bottom surface of said locking piece and each of said convex upper surfaces of said extensions, wherein said splines on said concave surfaces of said locking piece are configured to cooperate with and engage said splines on said convex upper surfaces of said extensions at said two or more positions corresponding to pivotal motion of said post portion.

17. The spinal implant system of claim 15, wherein said slot extends a sufficient length along said longitudinal axis to allow for lateral adjustment of said connector body relative to said bone screw, thereby allowing for positioning of said post portion of said bone screw at a plurality of positions along said slot.

18. The spinal implant system of claim 15, comprising a hole defined by said rod engaging portion in communication with said channel to secure a spinal rod in said channel.

19. A method, comprising:
providing a bone screw, wherein said bone screw has a threaded bone engaging portion, a threaded pivoting post portion, and a hinge portion between said bone engaging portion and said post portion, wherein said post portion is configured to pivot relative to said bone engaging portion, wherein said hinged portion includes two generally parallel side extensions extending from said bone engaging portion, each of said extensions having a generally convex upper surface;
engaging said bone engaging portion of said bone screw to a vertebra;
pivoting said post portion about said hinged portion to a desired angular position relative to said bone engaging portion;
providing a connector body having a longitudinal axis, and a rod engaging portion and a bone screw engaging portion, wherein said rod engaging portion defines a channel extending generally perpendicular to said longitudinal axis and configured for receipt of a spinal rod, and said adjustment portion defines a slot extending along said longitudinal axis and configured for receipt of said post portion of said fixation element;
positioning said connector body relative to said bone screw, wherein said post portion of said bone screw is received in said slot;
positioning said post portion at a desired position in said slot of said connector body
engaging a locking piece to said post portion of said bone screw, wherein said locking piece has a top and bottom surface and defines a hole therethrough, said post portion being received in said hole, wherein said bottom surface includes two generally parallel concave surfaces configured to engage said convex upper surfaces of said extensions at two or more positions; and
positioning said locking piece at a desired one of said two or more positions corresponding to said desired angular position.

20. The method of claim 19, comprising:
inserting a nut over said post portion of said bone screw; and
tightening said nut to lock said post portion at said desired angular position relative to said bone engaging portion and lock said connector body to said bone screw.

21. The method of claim 19, comprising positioning said locking piece over said post portion of said bone screw, wherein said post portion is received in said hole in said locking piece.

22. The method of claim 19, wherein said locking piece is positioned in said slot in said connector body, said connector body defining one or more grooves adjacent said slot and said locking piece having one or more extensions configured to be slidably received in said one or more grooves.

23. The method of claim 19, comprising positioning a spinal rod in said channel of said connector body.

24. The method of claim 23, wherein said connector body defines a hole in communication with said channel, the method further comprising inserting a retaining member in said hole and tightening said retaining member to secure said spinal rod in said channel.
